(19) United States
(12) Patent Application Publication
(10) Pub. No.: US 2015/0190607 A1
(43) Pub. Date: Jul. 9, 2015

(54) CONTROL METHOD, CONTROL DEVICE, AND EYE MASK

(71) Applicant: Panasonic Corporation, Osaka (JP)

(72) Inventors: Toshiyasu SUGIO, Osaka (JP); Masao KATO, Shiga (JP)

(21) Appl. No.: 14/309,940

(22) Filed: Jun. 20, 2014

(30) Foreign Application Priority Data
Jan. 9, 2014 (JP) 2014-002645

(51) Int. Cl. A61M 21/02; A61F 9/04

(52) U.S. Cl. CPC A61M 21/02; A61F 9/04; A61M 2021/0066

(57) ABSTRACT

A control method includes the steps of: (a) monitoring an electrooculogram signal of a user using an electrooculogram sensor for monitoring the electrooculogram signal of the user; (b) determining whether or not a sleep state of the user is a non-rapid eye movement (non-REM) sleep state, based on the electrooculogram signal monitored in step (a); and (c) performing a control operation to cause an element capable of warming the eye area of the user to warm an eye area of the user when the sleep state of the user is determined not to be the non-REM sleep state in step (b).

### CROSS REFERENCE TO RELATED APPLICATION

The present application is based on and claims priority of Japanese Patent Application No. 2014-002645 filed on Jan. 9, 2014. The entire disclosure of the above-identified application, including the specification, drawings and claims is incorporated herein by reference in its entirety.

### FIELD

The present disclosure relates to a control method, a control device, and an eye mask for assisting more efficient sleep of a user.

### BACKGROUND

An eye mask has been known which covers user's eyes and thus prevents outside light from reaching the user's eyes to assist user's sleep especially in well-lighted area. Moreover, Patent Literature 1 (Japanese Unexamined Patent Application Publication No. 2-280750) discloses such an eye mask that prevents light from reaching user's eyes. This also discloses that a far-infrared ceramic plate disposed at a position in an eye mask corresponding to user's eyes improves the flow of blood around the eyes to reduce eye strain and/or a feeling of fatigue in the entire head.

### SUMMARY

### Technical Problem

However, the above conventional techniques are not enough to assist more efficient sleep of a user.

Accordingly, one non-limiting and exemplary embodiment has been conceived in view of the foregoing disadvantages, and provides a control method and a control device for assisting more efficient sleep of a user.

### Solution to Problem

In order to solve the above problem, a control method according to an aspect of the present disclosure includes the steps of: (a) monitoring an electrooculogram signal of a user using an electrooculogram sensor for monitoring the electrooculogram signal of the user; (b) determining whether or not a sleep state of the user is a non-rapid eye movement (non-REM) sleep state, based on the electrooculogram signal monitored in step (a); and (c) performing a control operation to cause an element capable of warming the eye area of the user to warm an eye area of the user when the sleep state of the user is determined not to be the non-REM sleep state in step (b).

General and specific aspect disclosed above may be implemented using a control device, a system, an integrated circuit, a computer program, or a computer-readable recording medium such as a CD-ROM, or any combination of control devices, systems, integrated circuits, computer programs, or computer-readable recording media. Furthermore, the general and specific aspect may be implemented using an eye mask including the control device.

Additional benefits and advantages of the disclosed embodiments will be apparent from the Specification and Drawings. The benefits and/or advantages may be individually obtained by the various embodiments and features of the Specification and Drawings, which need not all be provided in order to obtain one or more of such benefits and/or advantages.

### Advantageous Effects

A control method, a control device, and an eye mask disclosed herein provide more efficient sleep to a user.

### BRIEF DESCRIPTION OF DRAWINGS

These and other objects, advantages and features of the disclosure will become apparent from the following description thereof taken in conjunction with the accompanying drawings that illustrate a specific embodiment of the present disclosure.

FIG. 1 illustrates an outline perspective view showing an outline of an eye mask according to an embodiment 1.

FIG. 2 illustrates a schematic view for describing positions of parts in the eye mask according to the embodiment 1 when the eye mask is mounted on a head of a user.

FIG. 3 illustrates an exploded perspective view of the eye mask according to the embodiment 1.

FIG. 4 illustrates a block diagram showing the functionality configuration of a control device according to the embodiment 1.

FIG. 5 illustrates a flow chart showing the procedure of a control method performed by the control device according to the embodiment 1.

FIG. 6 illustrates a comparison diagram of sleep level transitions and core body temperature transitions for a good sleeper and a bad sleeper.

FIG. 7 illustrates a block diagram showing the functionality configuration of a control device according to an embodiment 2.

FIG. 8 illustrates a flow chart showing the procedure of a control method performed by the control device according to the embodiment 2.

FIG. 9 illustrates a diagram showing the configuration of a control system according to the embodiment 3.

FIG. 10 illustrates a flow chart showing the procedure of a control method performed by a control device according to the embodiment 4.

### DESCRIPTION OF EMBODIMENTS

### (Underlying Knowledge Forming Basis of the Present Disclosure)

In relation to the eye mask disclosed in the Background section, the inventors have found the problem indicated below.

People are likely to sleep deeply when the temperature inside the body (hereinafter, referred to as “core body temperature”) is decreased. The core body temperature can be decreased by increasing the temperature of skin surrounding the body surface (hereinafter, referred to as “skin temperature”). This may be because the warmed body surface causes capillaries to expand in the vicinity of the body surface and thus the heat inside the body is easily released to the outside.

The conventional eye mask, however, fails to take into account the control of the core body temperature. In other words, the conventional eye mask can not control the core body temperature. Accordingly, it is difficult to efficiently assist the sleep of a user.

The present disclosure has been conceived from such knowledge, and provides a control method, a control device, and an eye mask for assisting more efficient sleep of a user as a result of the earnest study of the inventors.

In order to solve the aforementioned problem, a control method according to an aspect of the present disclosure includes the steps of: (a) monitoring an electrooculogram signal of a user using an electrooculogram sensor for monitoring the electrooculogram signal of the user; (b) determining whether or not a sleep state of the user is a non-rapid eye movement (non-REM) sleep state, based on the electrooculogram signal monitored in step (a); and (c) performing a control operation to cause an element capable of warming the eye area of the user to warm an eye area of the user when the sleep state of the user is determined not to be the non-REM sleep state in step (b).

With this, the electrooculogram signal is monitored to determine whether or not the sleep state of the user is the non-REM sleep state. If it is determined not to be the non-REM sleep, then the element warms the eyes. In general, the human's sleep state transitions to a deep sleep state (non-REM sleep state) by decreasing the core body temperature. This control method can increase the skin temperature over the whole body by warming the eye area where relatively more blood vessels exist than other body parts. As a result, the core body temperature can be decreased. Thus, when the sleep state of the user is determined not to be the non-REM sleep state, the element warms the eye area of the user to decrease the user's core body temperature, thereby achieving the sleep state transition of the user to a deep sleep state. Accordingly, it is possible for the user to sleep efficiently.

Furthermore, for example, in step (b), the sleep state of the user may be determined to be the non-REM sleep state when it is determined based on the electrooculogram signal that an eye movement of the user stops.

Furthermore, for example, in step (b), the sleep state of the user may be determined not to be the non-REM sleep state when it is determined based on the electrooculogram signal that the eye movement of the user occurs again after the eye movement of the user has stopped.

Furthermore, for example, the control method may further includes the step of (d) setting a sleep duration or a wake-up time, in which the element may be further capable of cooling the eye area, in step (b), the number of times that the sleep state may be determined not to be the non-REM sleep state is counted for one sleep period of the user, and in step (c), the element may cool the eye area when (i) a count of the number of times that the sleep state is determined not to be the non-REM sleep state for the one sleep period in step (b) is two or more, and (ii) the sleep duration set in step (d) has passed since the one sleep period started, or a current time is after the wake-up time set in step (d).

Furthermore, for example, in step (c), in the case where the element cools the eye area, the element may start to cool the eye area in a predetermined time after the sleep state is determined not to be the non-REM sleep state in step (b).

Furthermore, for example, the control method may further include the steps of: (e) setting one of a first mode and a second mode as a control mode in step (c); and (f) determining whether the control mode currently set in the second setting is the first mode or the second mode, in which in step (c), if the control mode is determined to be the first mode in step (f), then the element may cool the eye area when (i) the count of the number of times that the sleep state is determined not to be the non-REM sleep state for the one sleep period in step (b) is two or more, and (ii) the sleep duration set in step (d) has passed since the one sleep period started, or the current time is after the wake-up time set in step (d), and if the control mode is determined to be set to the second mode in step (e), then the element may cool the eye area when a preset sleep duration has passed.

Furthermore, for example, in step (c), assist control may be further performed to cause a peripheral apparatus disposed around the user to assist a transition of the sleep state of the user to the non-REM sleep state when the sleep state of the user is determined not to be the non-REM sleep state in step (b).

Furthermore, for example, the peripheral apparatus may be an aroma diffuser, and in step (c), the assist control may be performed by generating aroma for assisting the sleep of the user, from the aroma diffuser to assist sleep of the user, the aroma being.

Furthermore, for example, the aroma for assisting the sleep of the user may be lavender aroma.

Furthermore, for example, the control method may be implemented as a control device including: an electrooculogram sensor for monitoring an electrooculogram signal; an element capable of warming an eye area; and a control unit configured to control the element based on the electrooculogram signal monitored by the electrooculogram sensor, in which the control unit includes: an electrooculogram unit configured to monitor the electrooculogram signal using the electrooculogram sensor; a determination unit configured to determine whether or not a sleep state is a non-rapid eye movement (non-REM) sleep state, based on the electrooculogram signal; and an operation control unit configured to cause the element to warm the eye area when the determination unit determines that the sleep state is not the non-REM sleep state.

Furthermore, for example, the control method may be implemented as an eye mask covering eyes of a user when the eye mask is mounted on a head of the user, the eye mask including: a main body including the control device in an aspect of the present disclosure and a cover covering the eyes; and a holding part for holding the main body on the head.

Furthermore, for example, the electrooculogram sensor may be disposed in contact with skin around the eyes when the main body is held on the head by the holding part.

Furthermore, for example, the control unit may include a circuit board with circuitry providing functionality of the control unit, and the circuit board may be disposed at a position in the main body corresponding to middle of the eyes when the main body is held on the head by the holding part.

Furthermore, for example, the control device may further include a battery for supplying power to drive the circuit board, and the battery may be disposed at a position in the main body corresponding to the middle of the eyes when the main body is held on the head by the holding part.

These general and specific aspects may be implemented using a system, an integrated circuit, a computer program, or a computer-readable recording medium such as a CD-ROM, or any combination of systems, integrated circuits, computer programs, or computer-readable recording media.

Hereinafter, embodiments are specifically described with reference to the Drawings. Note that the excessively detailed explanation may be omitted. For example, the detailed explanation of a well-known matter or the same explanation for the substantially the same configuration may be omitted. This is to avoid the unnecessary redundancy in the following description and help those skilled in the art to understand the embodiments.

Hereinafter, a control device according to an exemplary embodiment of the present disclosure is described with reference to drawings, but the exemplary embodiment of the present disclosure does not limit this.

### Embodiment 1

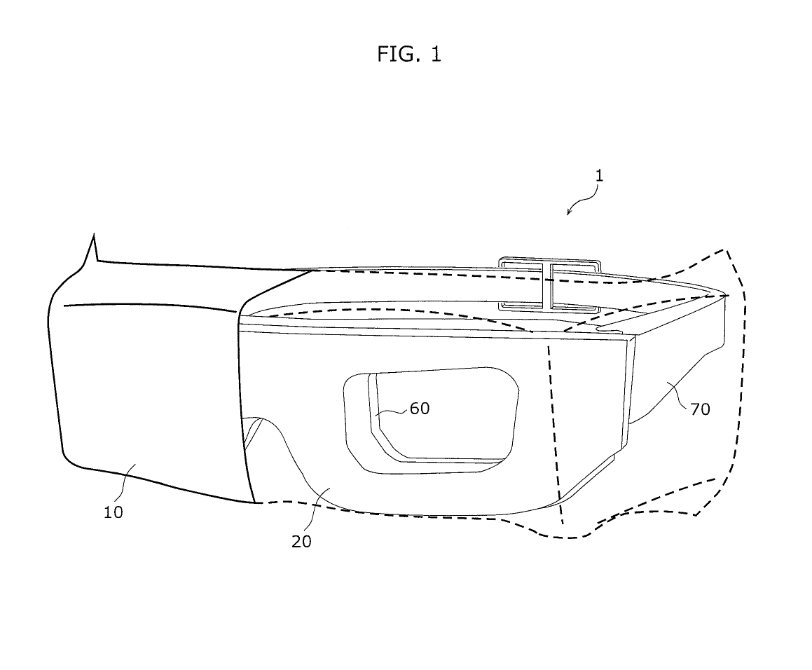

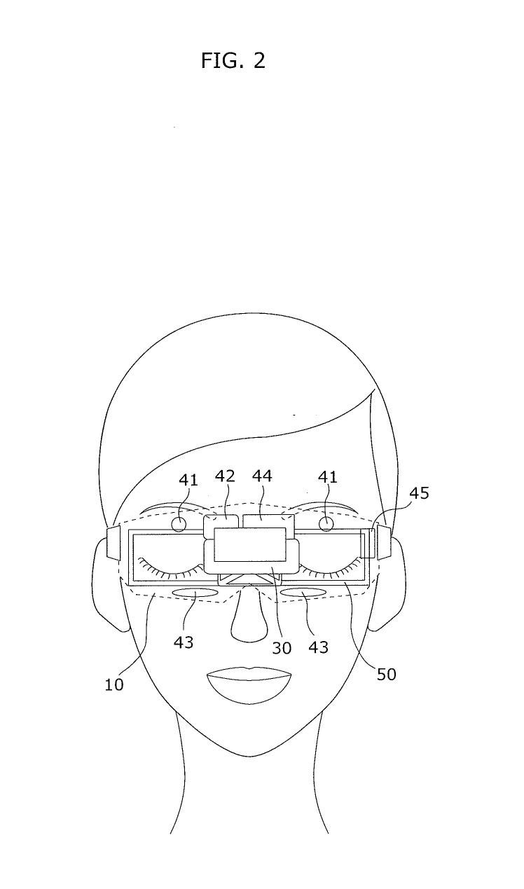

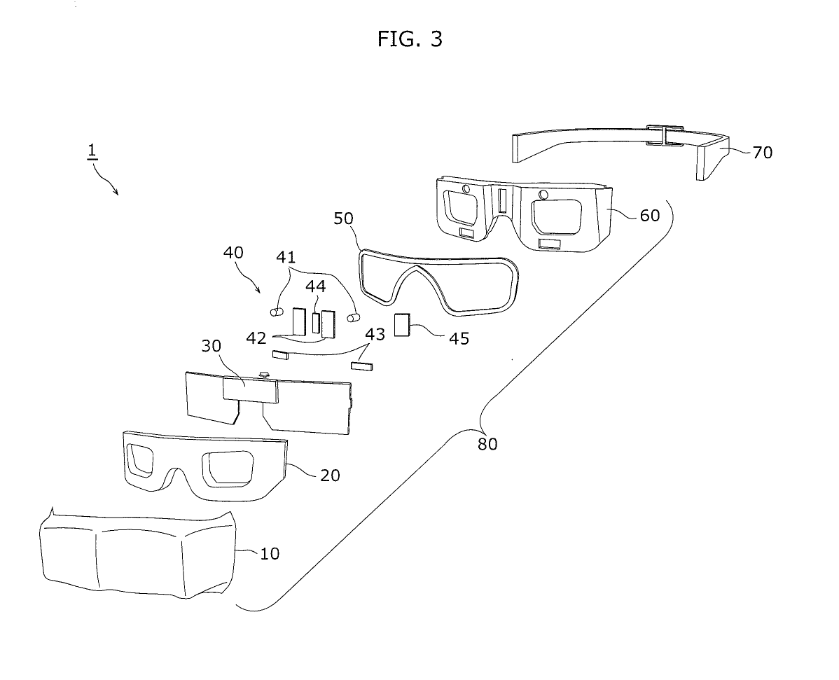

FIG. 1 illustrates an outline perspective view showing an outline of an eye mask according to an exemplary embodiment. FIG. 2 illustrates a schematic view for describing positions of parts in the eye mask according to the embodiment when the eye mask is mounted on a head of a user. FIG. 3 illustrates an exploded perspective view of the eye mask according to the embodiment.

As shown in FIG. 1 to FIG. 3, the eye mask 1 includes a main body 80 and a holding part 70. The main body 80 includes a cover 10, a housing 20, a battery 30, a control device 40, an electrical heating material 50, and a silicone rubber 60. The holding part 70 is a band-shaped member made of fabric and rubber. The both ends of the holding part 70 are respectively connected to the both ends of the main body 80. The holding part 70 is also a member placed around the side and back of a head to hold the main body 80 on a face side of the head when the eye mask 1 is mounted on the head.

The cover 10 is a resin cover covering eyes of the user when the eye mask 1 is mounted on the head. The cover 10 is also an outer member of the main body 80 in the eye mask 1. The housing 20 is a resin structure for keeping the eye mask 1 in shape fitting the periphery of the eyes in the head. The battery 30 supplies power to drive the control device 40 (especially, circuitry on a circuit board 44).

The control device 40 includes electrooculogram sensors 41, a Peltier element 42, a vibration element 43, the circuit board 44, and a communication board 45.

The electrooculogram sensor 41 detects a potential around an eye (electrooculogram signal). As shown in FIG. 2, the two electrooculogram sensors 41 are disposed in contact with skin around the eyes when the eye mask 1 is mounted on the head (i.e. when the main body 80 is held on the face side of the head by the holding part 70). In other words, each of the electrooculogram sensors 41 is disposed at a different one of the positions corresponding to the eyes. It is to be noted that although the electrooculogram sensor 41 is disposed between the eye and the eyebrow in FIG. 2, any position is possible as long as the electrooculogram sensor 41 is in contact with the skin around the eye. For example, the electrooculogram sensor 41 may be disposed in contact with the skin on the left or right of the eye, or under the eye.

The Peltier element 42 is an element capable of warming and cooling the periphery of the eyes through the electrical heating material 50 when the eye mask 1 is mounted on the head. The Peltier element 42 includes the first portion and the second portion (not shown), in which two states are switched by changing the direction of a current flow. One of the two states is a state where the first portion releases the heat (heating) and the second portion absorbs the heat (cooling), and the other is a state where the first portion absorbs the heat (cooling) and the second portion releases the heat (heating). The first portion of the Peltier element 42 is in contact with the electrical heating material 50.

The vibration element 43 is a piezoelectric element disposed in contact with the skin under the eye to vibrate the skin under the eye. It is to be noted that the vibration element 43 may use a vibration motor instead of the piezoelectric element.

The circuit board 44 has a central processing unit (CPU) and a memory mounted thereon (not shown), and drives by receiving power from the battery 30. The circuit board 44 also has circuitry for controlling the Peltier element 42 and the vibration element 43 according to the detection result from the electrooculogram sensor 41. The circuit board 44 is disposed on the center of the main body 80 where the bending stress applied to the housing 20 is the least. In other words, the circuit board 44 is disposed at a position in the main body 80 corresponding to the middle of the eyes when the main body 80 is held on the face side of the head by the holding part 70. It is to be noted that the battery 30 is disposed close to the circuit board 44. In other words, like the circuit board 44, the battery 30 is disposed at a position in the main body 80 corresponding to the middle of the eyes when the main body 80 is held on the face side of the head by the holding part 70.

The communication board 45 is a board with circuitry for communication via Bluetooth (registered trademark). The communication board 45 allows the eye mask 1 to communicate with an information terminal such as a smart phone via Bluetooth (registered trademark) for example. It is to be noted that the communication board 45 may be a board with circuitry for wireless communication via wireless LAN such as Wi-Fi.

The electrical heating material 50 is a metal material (for example, aluminum) for transferring the heat generated in the Peltier element 42 to the skin around eyes for heat release and transferring the heat of the skin around the eyes to the Peltier element 42 for heat absorption when the main body 80 is held on the face side of the head.

The silicone rubber 60 is a silicone-rubber cushion member placed in contact with the periphery of the eyes when the main body 80 is held on the face side of the head. In other words, the silicone rubber 60 is a member for reducing the stress from the main body 80 to the face caused by the eye mask 1 held on the head.

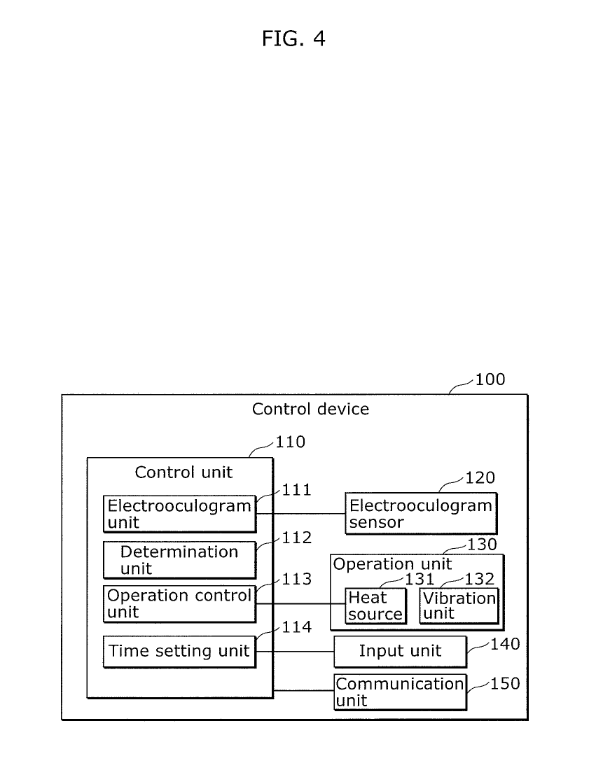

FIG. 4 illustrates a block diagram showing the functionality configuration of the control device according to the embodiment 1. In other words, FIG. 4 is a block diagram showing the configuration of functions of the control device 40 realized by the hardware configuration shown in FIG. 1 to FIG. 3.

The control device 100 includes a control unit 110, the electrooculogram sensor 120, an operation unit 130, an input unit 140, and a communication unit 150.

The electrooculogram sensor 120 is implemented as the electrooculogram sensor 41 shown in FIG. 2 and FIG. 3. The electrooculogram sensor 120 monitors the electrooculogram signal of the user.

The operation unit 130 includes a heat source 131 and a vibration unit 132. The heat source 131 is implemented as the Peltier element 42 shown in FIG. 2 and FIG. 3, and is capable of warming and cooling the eye area of the user (the periphery of the eyes in the user's face). The vibration unit 132 is implemented as the vibration element 43 shown in FIG. 2 and FIG. 3, vibration is applied to the eye area.

The input unit 140 is implemented as an input button or the like (not shown), and receives information indicating a sleep duration or a wake-up time from the user. The information received by the input unit 140 is set as the sleep duration or the wake-up time by a time setting unit 114.

The communication unit 150 is implemented as the communication board 45 shown in FIG. 3. The communication unit 150 transmits the result monitored by the electrooculogram sensor 120 by communicating with the information terminal such as a smart phone.

The control unit 110 is implemented as the circuit board 44 shown in FIG. 2 and FIG. 3, and controls the operation unit 130 according to the electrooculogram signal monitored by the electrooculogram sensor. The control unit 110 includes an electrooculogram unit 111, a determination unit 112, an operation control unit 113, and the time setting unit 114.

The electrooculogram unit 111 monitors the electrooculogram signal using the electrooculogram sensor 120. The determination unit 112 determines, based on the electrooculogram signal monitored by the electrooculogram sensor 120, whether or not a sleep state of a user is a non-REM sleep state. The operation control unit 113 activates the heat source 131 in the operation unit 130 to warm the eye area of the user when the determination unit 112 determines that the sleep state of the user is not the non-REM sleep state. It is to be noted that the case where the sleep state is determined not to be the non-REM sleep state is a case where the user is in the non-REM sleep state or a wakefulness state.

Furthermore, the determination unit 112 may determine that the sleep state of the user is the non-REM sleep state when it is determined based on the electrooculogram signal monitored by the electrooculogram sensor 120 that an eye movement of the user stops.

Furthermore, the determination unit 112 may determine that the sleep state of the user is not the non-REM sleep state when it is determined based on the electrooculogram signal monitored by the electrooculogram sensor 120 that the eye movement of the user occurs again after the eye movement of the user has stopped.

Specifically, the determination unit 112 determines that the eye movement of the user stops when the variation range of the electrooculogram signal monitored by electrooculogram sensor 120 is smaller than the predetermined range, while that the eye movement of the user occurs when the variation range of the electrooculogram signal is greater than the predetermined range.

The time setting unit 114 sets a sleep duration or a wake-up time. Specifically, the time setting unit 114 sets a sleep duration or a wake-up time provided from the user through the input unit 140. In this case, the determination unit 112 may count the number of times that the sleep state is determined not to be the non-REM sleep state for one sleep period of the user. Furthermore, in this case, the operation control unit 113 may cause the heat source 131 to cool the eye area when (i) a count of the number of times that the sleep state is determined not to be the non-REM sleep state for the one sleep period of the user is two or more, and (ii) the sleep duration set by the time setting unit 114 has passed since the one sleep period started, or a current time is after the wake-up time set by the time setting unit 114.

Furthermore, the communication unit 150 may receive information inputted through the information terminal. In other words, when the communication unit 150 receives information indicating a sleep duration or a wake-up time, the time setting unit 114 may set the sleep duration or the wake-up time received by the communication unit 150.

Furthermore, in the case where the heat source 131 cools the eye area, the operation control unit 113 may cause the heat source 131 to start to cool the eye area in a predetermined time after the determination unit 112 determines that the sleep state is not the non-REM sleep state.

Next, the procedure of the control method performed by the control device according to the foregoing embodiment is described.

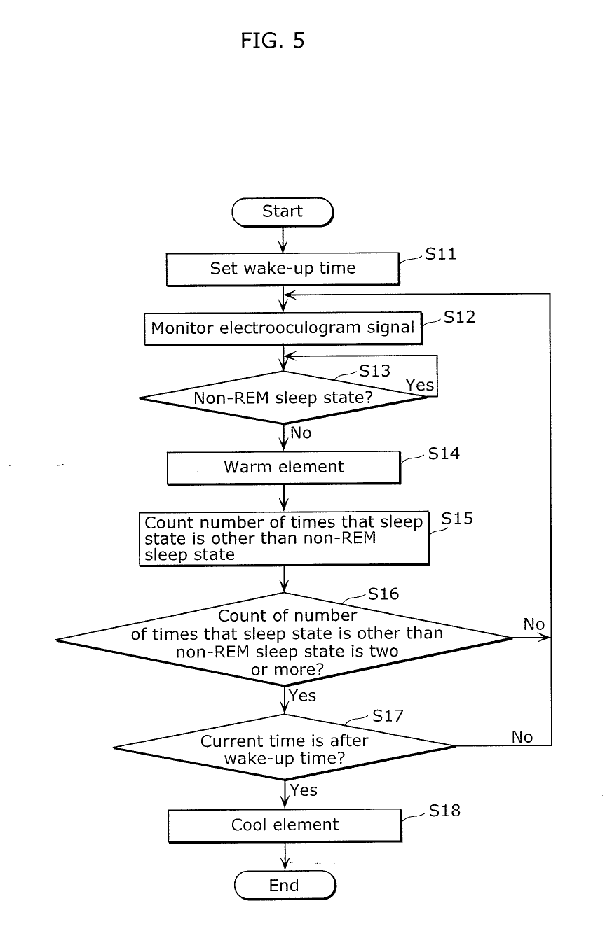

FIG. 5 illustrates a flow chart showing the procedure of the control method performed by the control device according to the embodiment 1.

First, the time setting unit 114 sets the wake-up time based on the information inputted through the input unit 140 or the information terminal such as a smart phone, operated by the user (S11). The time setting unit 114 may set the sleep duration based on the obtained information.

The electrooculogram unit 111 monitors the electrooculogram signal using the electrooculogram sensor 120 (S12: monitoring step).

The determination unit 112 determines whether or not the sleep state of the user is the non-REM sleep state based on the electrooculogram signal monitored in the monitoring step (S13: first determining step).

If the sleep state of the user is determined to be non-REM sleep state in the first determining step (Yes in S13), then the control unit 110 repeats the first determining step. In other words, the control unit 110 waits until the sleep state of the user is determined not to be the non-REM sleep state. It is to be noted that in Step S13, if (i) the sleep state of the user is determined to be the non-REM sleep state (Yes in S13) and (ii) the heat source 131 is warming the eye area of the user, then the heat source 131 may stop warming the eye area because the sleep state of the user is in a deep sleep level. In this way, it is possible to save the electric power of the battery 30.

In the first determination step, if the sleep state of the user is determined not to be the non-REM sleep state (No in S13), then the operation control unit 113 in the control unit 110 causes the heat source 131 (Peltier element 42) to warm the eye area of the user (S14: control step). In other words, in this case, the operation control unit 113 switches the mode of the heat source 131 to a mode in which heat is released from the first portion in the Peltier element 42 placed in contact with the electrical heating material 50.

Next, the determination unit 112 counts the number of times that the sleep state is determined not to be the non-REM sleep state (S15). Specifically, the determination unit 112 increments by one a count of the number of times that the sleep state is determined not to be the non-REM sleep state (hereinafter, referred to as “count”). It is to be noted that the count is 0 at the start of processing of the control method.

Next, the determination unit 112 determines whether or not the count of the number of times that the sleep state is determined not to be the non-REM sleep state is two or more (S16). In other words, the determination unit 112 determines whether or not the count is at least two.

When the count is determined to be two or more (Yes in S16), the determination unit 112 determines whether or not a current time is after the wake-up time (S17). In this case (Yes in S16), when a sleep duration is set by time setting unit 114, the determination unit 112 may determines whether or not the sleep duration set by the time setting unit 114 has passed since one sleep period of the user started. It is to be noted that the start time of the one sleep period may be a power-on time of the control device 100 by the user, a start time of Step S11, or a time when the electrooculogram sensor 120 starts to detect the electrooculogram signal.

If the determination unit 112 determines that the current time is after the wake-up time, or that the sleep duration has passed since the one sleep period of the user started (hereinafter, in both cases, referred to as “the current time is after the wake-up time”) (Yes in S17), then the operation control unit 113 switches the mode of the heat source 131 to the mode in which heat is absorbed into the first portion in the Peltier element 42, and processing ends (S18). It is to be noted that the operation control unit 113 may cause the vibration unit 132 to vibrate at the same time. Furthermore, at least one of the actions of the operation control unit 113, i.e. switching the mode of the heat source 131 to the mode in which heat is absorbed into the first portion in the Peltier element 42 and/or causing the vibration unit 132 to vibrate, may be performed in a predetermined time after the wake-up time, instead of immediately after the wake-up time. In this way, when (i) the count of the number of times that the sleep state is other than the non-REM sleep state is two or more, and (ii) the current time is after the wake-up time set by the time setting unit 114, at least one of the actions, i.e. the heat absorption into the electrical heating material 50 and/or the vibration of the vibration unit 132, is performed. Accordingly, it is possible to awake the user at an appropriate timing. After Step S18, the processing of the control method ends.

It is to be noted that the determination unit 112 determines whether or not a current time is after the wake-up time when the count is two or more, but the count is not limited to the two or more. Any count such as three or more and four or more is possible. The count may be at least a predetermined number.

In contrast, if it is determined that the count of the number of times that the sleep state is other than the non-REM sleep state is less than two (No in S16), or if it is determined that a current time is before the wake-up time (No in S17), then processing returns to Step S12.

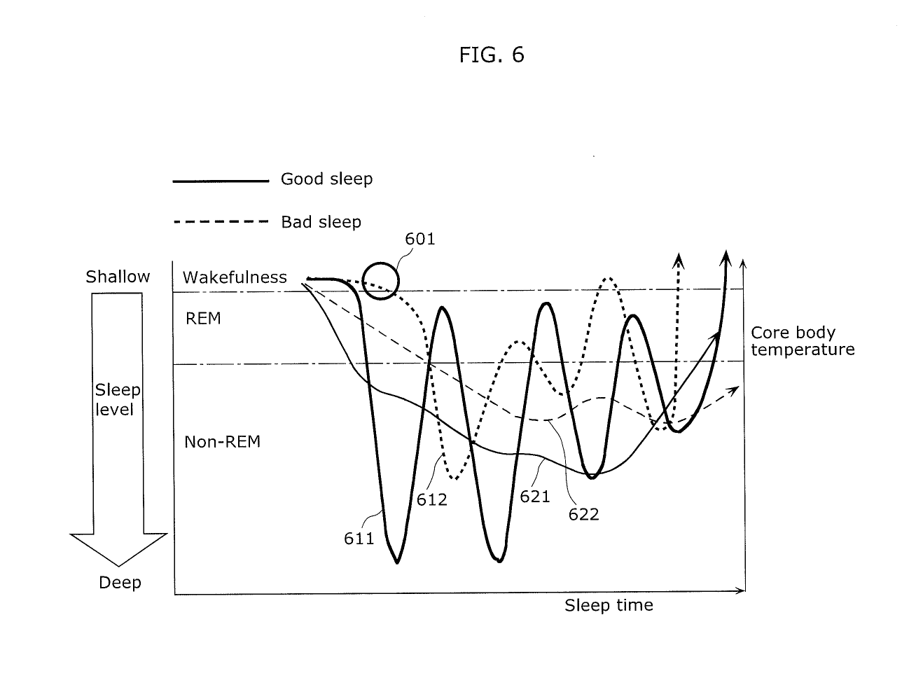

FIG. 6 illustrates a comparison diagram of sleep level transitions and core body temperature transitions for a good sleeper and a bad sleeper. In FIG. 6, the solid arrow indicates a good sleeper, while the dotted arrow indicates a bad sleeper. The heavy-line arrow indicates the sleep level transition, while the light-line arrow indicates the core body temperature transition. In other words, the arrow 611 indicates the sleep level transition of the good sleeper, the arrow 612 indicates the sleep level transition of the bad sleeper, the arrow 621 indicates the core body temperature transition of the good sleeper, and the arrow 622 indicates the core body temperature transition of the bad sleeper.

As shown in FIG. 6, the sleep state of the good sleeper transitions from the wakefulness state to the REM sleep state, and further a transition to the non-REM sleep state, which is a deeper sleep state, occurs earlier than that for the bad sleeper. For the good sleeper, the non-REM sleep state and the REM sleep state are alternately repeated multiple times, and the deepest sleep level during one non-REM sleep state gradually becomes shallow. Finally, the sleeper awakes. On the other hand, for the bad sleeper, although the non-REM sleep state and the REM sleep state are alternately repeated multiple times like the good sleeper, shallow sleep is repeated even when the sleeper is in the non-REM sleep state, or the sleeper awakes during the sleep period. Finally, the sleeper awakes. Thus, the sleep level of the bad sleeper tends to be shallower than that of the good sleeper over the whole sleep period.

In the comparison of core body temperature between the good sleeper and the bad sleeper, the core body temperature of the good sleeper decreases sharply immediately after the start of the sleep period, whereas the core body temperature of the bad sleeper decreases more moderately than that of the good sleeper even when the sleep period starts. The lowest core body temperature of the bad sleeper during one sleep period is higher than that of the good sleeper. Thus, the bad sleeper tends to be hard to decrease his/her core body temperature even when the sleep period starts, and a transition to a deep sleep state hardly occurs.

When such a bad sleeper wears the eye mask 1 according to the embodiment 1 and sleeps, the Peltier element 42 in the eye mask 1 is controlled to warm the eye area during the sleep state other than the non-REM sleep state. For example, the eye area can be warmed during the state 601 shown in FIG. 6. In this way, the sleep state can be transitioned to a state in which his/her core body temperature easily decreases, and it is possible to bring the sleep state of the bad sleeper close to that of the good sleeper. This assists the more efficient sleep.

According to the control method in the embodiment 1, the determination unit 112 in the control unit 110 determines whether or not the sleep state of the user is the non-REM sleep state by monitoring the electrooculogram signal of the user. If the determination unit 112 determines that the sleep state is not the non-REM sleep, then the operation control unit 113 causes the Peltier element 42 to warm the eye area. In general, a human's sleep state transitions to a deep sleep state (non-REM sleep state) by decreasing the core body temperature. This control method can increase the skin temperature over the whole body by warming the eye area where relatively more blood vessels exist than other body parts. As a result, the core body temperature can be decreased. Thus, when the sleep state of the user is determined not to be the non-REM sleep state, the eye area of the user is warmed using the heat generated by the Peltier element 42, thereby decreasing the core body temperature of the user. In this way, the sleep state of the user can transition to the deep sleep state. Accordingly, it is possible for the user to sleep efficiently.

### Embodiment 2

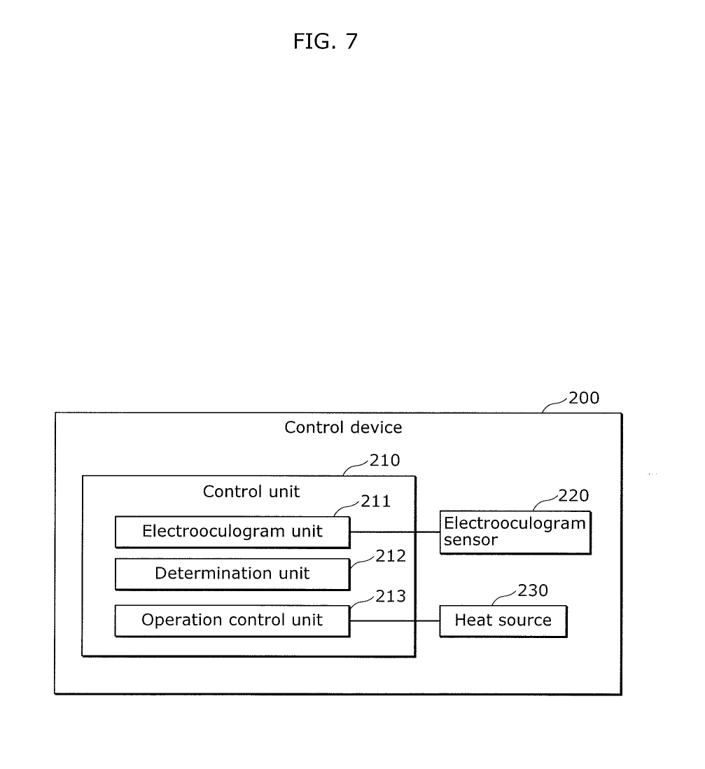

FIG. 7 illustrates a block diagram showing the functionality configuration of a control device according to an embodiment 2.

In the embodiment 1, the Peltier element 42 is used as the heat source 131, and the Peltier element 42 cools the eye area of the user at the wake-up time set by the time setting unit 114. Such a structure, however, is not necessarily required.

The control device 200 includes a control unit 210, the electrooculogram sensor 220, and a heat source 230. In other words, the time setting unit 114, the vibration unit 132, the input unit 140, and the communication unit 150 in the embodiment 1 are not required. The electrooculogram unit 211 has the same structure as the electrooculogram unit 111 in the embodiment 1. The electrooculogram sensor 220 also has the same structure as the electrooculogram sensor 120 in the embodiment 1. Furthermore, a determination unit 212 differs from the determination unit 112 according to the embodiment 1 in that the number of times that the sleep state is determined not to be the non-REM sleep state is not counted. Furthermore, an operation control unit 213 differs from the operation control unit 113 in the embodiment 1 in that only the heat source 230 is controlled. Furthermore, a heat source 230 need not have a cooling function like the Peltier element 42. For example, it is possible to use a heating element such as a heating wire.

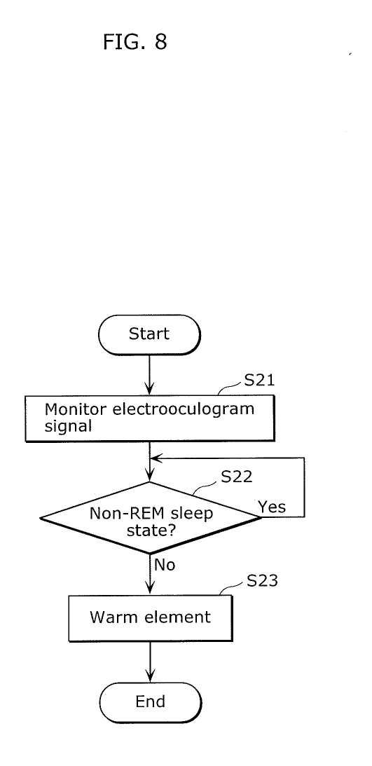

FIG. 8 illustrates a flow chart showing the procedure of a control method performed by the control device according to the embodiment 2.

First, the electrooculogram unit 211 monitors an electrooculogram signal using the electrooculogram sensor 220 (S21: monitoring step).

Next, the determination unit 212 determines whether or not the sleep state of a user is a non-REM sleep state based on the electrooculogram signal monitored in the monitoring step (S22: first determining step).

If the sleep state of the user is determined to be non-REM sleep state in the first determining step (Yes in S22), then the control unit 210 repeats the first determining step. In other words, the control unit 210 waits until the sleep state of the user is determined not to be the non-REM sleep state.

If the sleep state of the user is determined not to be the non-REM sleep state in the first determination step (No in S22), then the operation control unit 213 in the control unit 210 causes the heat source 131 to warm the eye area of the user (S23: control step), and processing of the control method ends.

### Embodiment 3

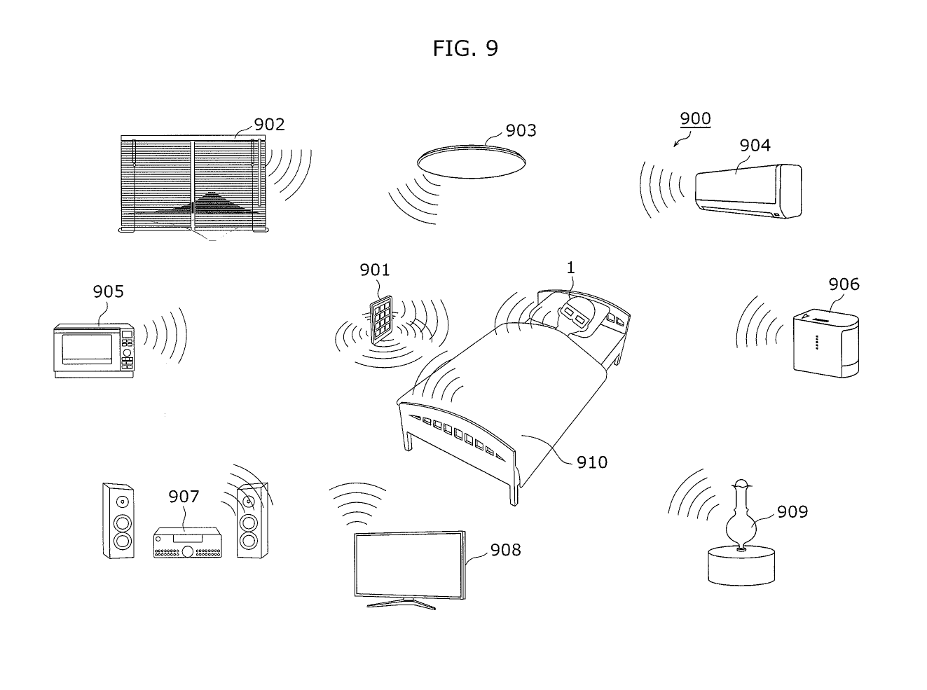

FIG. 9 illustrates a diagram showing the configuration of a control system according to an embodiment 3.

The control system 900 includes the eye mask 1 described in the embodiment 1, an information terminal 901 such as a smart phone, and peripheral apparatuses 902 to 910. The peripheral apparatuses 902 to 910 include a window shade 902, a lighting apparatus 903, an air conditioner 904, a microwave 905, a humidifier 906, an audio apparatus 907, a television 908, an aroma diffuser 909, and a blanket 910 capable of heating by a heating wire. The peripheral apparatuses 902 to 910 are communicatable with the information terminal 901 via Bluetooth (registered trademark) or Wi-Fi. Upon receiving a control instruction from the information terminal 901, each of the peripheral apparatuses 902 to 910 performs the control operation according to the received control instruction.

In the control devices 100 and 200 according to the embodiments 1 and 2, only the eye mask 1 is used to perform the control operation to assist a sleep state transition of a user. However, the information terminal 901 may obtain the user's sleep state determined by the eye mask 1 to cause the peripheral apparatuses 902 to 910 to perform various control operations according to the determined sleep state.

Specifically, upon receiving sleep information indicating a user's sleep state determined by the determination unit 112 in the eye mask 1, the information terminal 901 may cause the peripheral apparatuses 902 to 910 to perform various control operations to assist the sleep state transition of the user to the non-REM sleep state, for example, in Step S14 shown in FIG. 5, when the received sleep information indicates that the user's sleep state is not the non-REM sleep state. The information terminal 901 may also cause the peripheral apparatuses 902 to 910 to perform various control operations to assist the transition from a sleep state to a wakefulness state, for example, in Step S18 shown in FIG. 5, when (i) the count of the number of times that the sleep state is other than the non-REM sleep state during one sleep period of the user is at least a predetermined number and (ii) a current time is after the wake-up time.

For example, the various control operations to assist the sleep state transition of the user to the non-REM sleep state are the following operations of: for the lighting apparatus 903, reducing the illuminance of the lighting apparatus 903, i.e. changing the color of the lighting apparatus 903 to a color that assists the sleep; for the air conditioner 904, performing a heating operation to warm the user's body; for the humidifier 906, humidifying a sleeping space of the user to an appropriate humidity; for the audio apparatus 907, reproducing classical music for relaxing the user to assist the sleep; for the television 908, reproducing a video (for example, images of natural scenes) and music (for example, classical music, sound of nature, sound of rain, or sound of surf) for relaxing the user to assist the sleep; for the aroma diffuser 909, generating aroma for assisting the sleep (for example, lavender aroma); and for the blanket 910, warming the user's body using the heat generated from the heating wire.

Furthermore, for example, the various control operations to assist the transition from a sleep state to a wakefulness state are the following operations of: for the window shade 902, opening the window shade 902 or adjusting the angle of the window shade 902 so that the light enters into the sleeping space of the user through the widow; for the lighting apparatus 903, emitting white light; for the air conditioner 904, performing a cooling operation or a fan operation to cool the user's body; for the microwave 905, generating a sound to awake the user; for the humidifier 906, stopping humidifying the sleeping space of the user; for the audio apparatus 907, reproducing music or sound for assisting the wakefulness of the user; for the television 908, reproducing a video for assisting the wakefulness of the user; and for the blanket 910, stopping warming the user's body.

Furthermore, as one of the various control operations to assist the sleep state transition of the user to the non-REM sleep state, for example, the information terminal 901 may perform a control operation of reproducing classical music for relaxing the user to assist the sleep. Furthermore, as one of the various control operations to assist the transition from a sleep state to a wakefulness state, for example, the information terminal 901 may perform a control operation of generating an alarm sound to awake the user. In other words, the information terminal 901 may not only cause the peripheral apparatuses to perform the various control operations according to the sleep state of the user in conjunction with the peripheral apparatuses, but also perform by itself a control operation of giving direct action to the user's senses according to the sleep state of the user.

### Embodiment 4

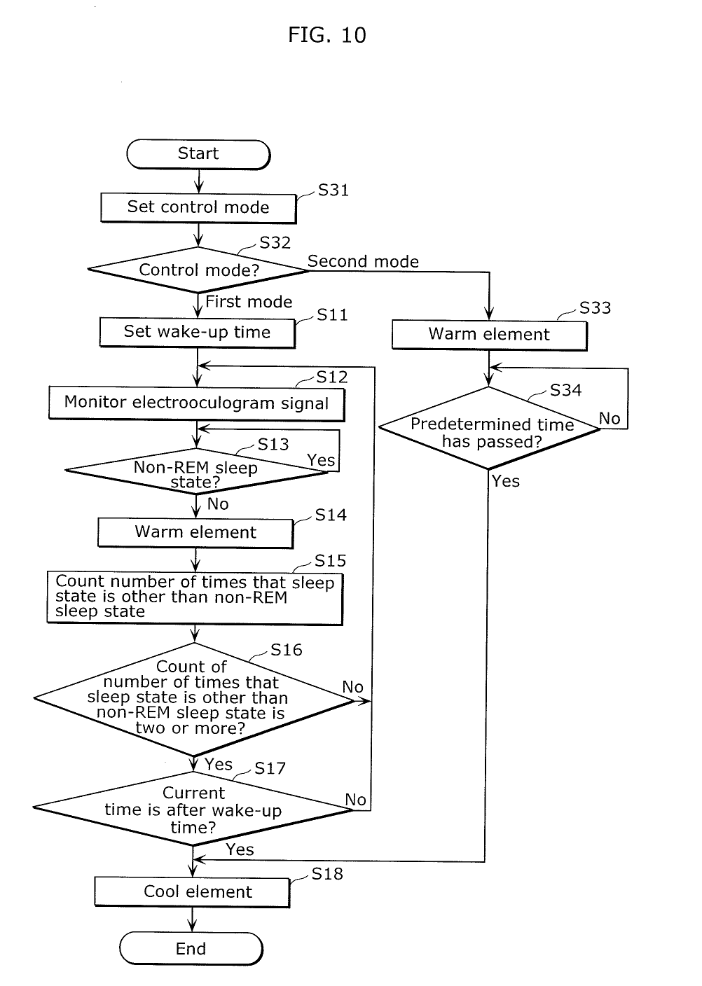

A control method performed by a control device according to an embodiment 4 is described. The control method performed by the control device according to the embodiment 4 differs from the control method in the embodiment 1 in that two control modes exist. Specifically, there are two control modes: one is the first mode intended to get adequate sleep; and the other is the second mode intended to take a short sleep (for example, 10 to 30 minutes), and each of the control modes has a different timing for awaking the user. The control method performed by the control device according to the embodiment 4 is described with reference to FIG. 10. It is to be noted that the control device according to the embodiment 4 is the same as the control device 100 according to the embodiment 1.

FIG. 10 is a flow chart showing the procedure of the control method performed by the control device according to the embodiment 4.

First, the control unit 110 sets the control mode based on information inputted through the input unit 140 or the information terminal such as a smart phone, operated by the user (S31: second setting step).

Next, the control unit 110 determines whether the set control mode is the first mode or the second mode (S32: second determining step).

If the control mode is determined to be the first mode (the first mode in S32), then the control unit 110 performs the steps S11 to S18 described in the embodiment 1. It is to be noted that the steps S11 to S18 are the same as those in the embodiment 1. Accordingly, their descriptions are omitted.

In contrast, if the control mode is determined to be the second mode (the second mode in S32), then like Step S14, the operation control unit 113 of the control unit 110 causes the heat source 131 (Peltier element 42) to warm the eye area of the user (S33: control step).

The determination unit 112 determines whether or not a preset sleep duration has passed since Step S32 was performed or Step S33 was started (S34). It is to be noted that the preset sleep duration is a sleep duration in the second mode which is set to 10 to 30 minutes for example. The sleep duration may be set to a predetermined duration by default, or may be changed by the user. For the sake of taking a short sleep, the sleep duration may have a settable upper limit (for example, 30 minutes) when the change by the user is available.

If it the determination unit 112 determines that the preset sleep duration has passed since Step S32 was performed or Step S33 was started (Yes in S34), then Step S18 in the embodiment 1 is performed, and processing ends.

In contrast, if it is determined that the sleep duration has not yet passed (No in S34), then processing returns to Step S34.

The control method performed by the control device 100 according to the embodiment 4 has two control modes, i.e. the first mode and the second mode. Accordingly, even when the user desires to take a short sleep such as a nap, the control operation is performed to assist the sleep and awake the user at the setting time. In this way, it is possible to perform the control operation to assist the sleep or wakefulness at an appropriate timing according to the user's state.

### (Variations)

(1)

In the embodiments 1 to 3, the electrooculogram signal of a user is used to determine whether or not to be non-REM sleep state, but any other means may be used. For example, the eye area of a user may be captured by a camera to determine whether or not an eye movement occurs based on the analysis of the captured images. When the eye stops, the sleep state is determined to be non-REM sleep state. Alternatively, for example, the sleep state may be determined to the non-REM sleep state when a user's body stops. In this case, the body movement of a user may be determined in the following way: the user's body is captured with a camera and the captured images are analyzed to determine whether or not the body movement occurs; the user's body is irradiated with radio waves to measure the distance to the user's body, and when the resulting distances are constant, it is determined that no body movement of the user occurs; or an acceleration sensor is disposed at a position where the body movement of the user can be detected, e.g. near the user's body or by user's bedside, and it is determined whether or not the body movement of the user occurs based on the result from the acceleration sensor. Alternatively, for example, electrodes for monitoring brain waves may be disposed on a user's head to determine whether or not to be the non-REM sleep state based on the brain waves. Alternatively, for example, electrodes for monitoring an electromyogram signal of a user may be disposed to determine whether or not to be the non-REM sleep state based on the electromyogram signal.

(2)

In the embodiments 1 to 3, the control devices 100 and 200 cause the heat source 131 to warm the eye area of a user for a sleep state transition of the user to a deep sleep state, but any other means may be used. For example, the control device may further include an aroma generation device and a music reproduction device. In this case, the control device may not only cause the heat source 131 to warm the eye area of the user, but also activate the aroma generation device which generates aroma for assisting the sleep of the user (for example, lavender aroma), and may further reproduce music for assisting the sleep of the user from a speaker. Instead of the warming of the eye area, it is possible to generate aroma, or reproduce music.

(3)

In the embodiment 3, upon receiving sleep information indicating a user's sleep state determined by the determination unit 112 in the eye mask 1, the information terminal 901 causes the peripheral apparatuses 902 to 910 to perform various control operations according to the received sleep information, but any other means can be used. For example, the information terminal 901 may receive information indicating the result from the electrooculogram sensor 120 to determine the sleep state of a user. In other words, the information terminal 901 may serve as the determination unit 112, the operation control unit 113 (which controls the peripheral apparatuses 902 to 910 in this case), and the time setting unit 114 in the control unit 110.

Alternatively, the information terminal 901 may manage the sleep state of a user by receiving sleep information indicating the sleep state determined in the determination unit 112 of the eye mask 1 and accumulating the received sleep information. Specifically, the information terminal 901 may provide a visual representation in which the sleep state indicated by the obtained sleep information is associated with the date and time of the sleep (for example, the graph showing the relation between the sleep time and the sleep level as shown in FIG. 6, or a visual representation of user's sleep quality). Alternatively, the information terminal 901 may analyze the obtained sleep information and display a message to improve the user's sleep quality.

(4)

In the embodiment 1, the holding part 70 in the eye mask 1 is a band-shaped member wrapped around the head of a user to hold the main body 80 at the eye position on a face side of the head. For example, the holding part 70 may be a member having a shape horizontally extending from the both ends of the main body 80 along the side of a head, and sandwiches the head at the both sides of the head to hold the main body 80.

(5)

In the embodiment 1, the circuit board 44 in the eye mask 1 is driven by power from the battery 30, but may be driven by power obtained by connecting to a utility power source.

Although only some exemplary embodiments have been described above, the scope of the Claims of the present application is not limited to these embodiments. Those skilled in the art will readily appreciate that various modifications may be made in these exemplary embodiments and that other embodiments may be obtained by arbitrarily combining the structural elements of the embodiments without materially departing from the novel teachings and advantages of the subject matter recited in the appended Claims. Accordingly, all such modifications and other embodiments are included in the present disclosure.

### INDUSTRIAL APPLICABILITY

The present disclosure is useful as an eye mask capable of performing a control operation to assist efficient sleep of a user.

1. A control method comprising the steps of:
(a) monitoring an electrooculogram signal of a user using an electrooculogram sensor for monitoring the electrooculogram signal of the user;
(b) determining whether or not a sleep state of the user is a non-rapid eye movement (non-REM) sleep state, based on the electrooculogram signal monitored in step (a); and
(c) performing a control operation to cause an element to warm an eye area of the user when the sleep state of the user is determined not to be the non-REM sleep state in step (b), the element being capable of warming the eye area of the user.

2. The control method according to claim 1,
wherein in step (b), the sleep state of the user is determined to be the non-REM sleep state when it is determined based on the electrooculogram signal that an eye movement of the user stops.

3. The control method according to claim 2,
wherein in step (b), the sleep state of the user is determined not to be the non-REM sleep state when it is determined based on the electrooculogram signal that the eye movement of the user occurs again after the eye movement of the user has stopped.

4. The control method according to claim 3, further comprising the step of
(d) setting a sleep duration or a wake-up time,
wherein the element is further capable of cooling the eye area,
in step (b), the number of times that the sleep state is determined not to be the non-REM sleep state is counted for one sleep period of the user, and
in step (c), the element cools the eye area when (i) a count of the number of times that the sleep state is determined not to be the non-REM sleep state for the one sleep period in step (b) is two or more, and (ii) the sleep duration set in step (d) has passed since the one sleep period started, or a current time is after the wake-up time set in step (d).

5. The control method according to claim 4,
wherein in step (c), in the case where the element cools the eye area, the element starts to cool the eye area in a predetermined time after the sleep state is determined not to be the non-REM sleep state in step (b).

6. The control method according to claim 5, further comprising the steps of:
(e) setting one of a first mode and a second mode as a control mode in step (c); and
(f) determining whether the control mode currently set in the second setting is the first mode or the second mode,
wherein in step (c),
if the control mode is determined to be the first mode in step (f), then the element cools the eye area when (i) the count of the number of times that the sleep state is determined not to be the non-REM sleep state for the one sleep period in step (b) is two or more, and (ii) the sleep duration set in step (d) has passed since the one sleep period started, or the current time is after the wake-up time set in step (d), and
if the control mode is determined to be set to the second mode in step (e), then the element cools the eye area when a preset sleep duration has passed.

7. The control method according to claim 6,
wherein in step (c), assist control is further performed to cause a peripheral apparatus to assist a transition of the sleep state of the user to the non-REM sleep state when the sleep state of the user is determined not to be the non-REM sleep state in step (b), the peripheral apparatus being disposed around the user.

8. The control method according to claim 7,
wherein the peripheral apparatus is an aroma diffuser, and
in step (c), the assist control is performed by generating aroma from the aroma diffuser to assist sleep of the user, the aroma being for assisting the sleep of the user.

9. The control method according to claim 8,
wherein the aroma for assisting the sleep of the user is lavender aroma.

10. A control device comprising:
an electrooculogram sensor for monitoring an electrooculogram signal;
an element capable of warming an eye area; and
a control unit configured to control the element based on the electrooculogram signal monitored by the electrooculogram sensor,
wherein the control unit includes:
an electrooculogram unit configured to monitor the electrooculogram signal using the electrooculogram sensor;
a determination unit configured to determine whether or not a sleep state is a non-rapid eye movement (non-REM) sleep state, based on the electrooculogram signal; and
an operation control unit configured to cause the element to warm the eye area when the determination unit determines that the sleep state is not the non-REM sleep state.

11. An eye mask covering eyes of a user when the eye mask is mounted on a head of the user, the eye mask comprising:
a main body including the control device according to claim 10 and a cover covering the eyes; and
a holding part for holding the main body on the head.

12. The eye mask according to claim 11,
wherein the electrooculogram sensor is disposed in contact with skin around the eyes when the main body is held on the head by the holding part.

13. The eye mask according to claim 12,
wherein the control unit includes a circuit board with circuitry providing functionality of the control unit, and
the circuit board is disposed at a position in the main body corresponding to middle of the eyes when the main body is held on the head by the holding part.

14. The eye mask according to claim 13,
wherein the control device further includes a battery for supplying power to drive the circuit board, and
the battery is disposed at a position in the main body corresponding to the middle of the eyes when the main body is held on the head by the holding part.
